(19) United States
(12) Patent Application Publication
(10) Pub. No.: US 2020/0009270 A1
(43) Pub. Date: Jan. 9, 2020

(54) OPHTHALMIC SOLUTION OF LISSAMINE GREEN AND ITS USE IN OPHTHALMOLOGY

(71) Applicant: OFTALAB SRL, Milano (IT)

(72) Inventor: Stefano Barabino, Milano (IT)

(73) Assignee: OFTALAB SRL, Milano (IT)

(21) Appl. No.: 16/469,662

(22) PCT Filed: Dec. 14, 2017
(86) PCT No.: PCT/IB2017/057935

§ 371 Date: Jun. 14, 2019

(30) Foreign Application Priority Data
Dec. 15, 2016 (IT) 102016000127037

(51) Int. Cl. A61K 49/00; A61K 47/12; A61K 47/02; A61K 47/18; A61K 47/36; A61K 47/32; A61K 47/26; A61K 9/00; A61K 9/08

(52) U.S. Cl. CPC A61K 49/006; A61K 47/12; A61K 47/02; A61K 47/183; A61K 9/08; A61K 47/32; A61K 47/26; A61K 49/0071; A61K 9/0048; A61K 47/36

(57) ABSTRACT

The present invention relates to an ophthalmic solution of lissamine green which has shown to be particularly stable over time and well tolerated by the eye. Therefore, the ophthalmic solution finds advantageous application in the diagnosis of corneal and conjunctival anomalies.

### FIELD OF INVENTION

The present invention relates to an ophthalmic solution of lissamine green which has shown to be particularly stable over time and well tolerated by the eye. Therefore, the ophthalmic solution finds advantageous application in the diagnosis of corneal and conjunctival anomalies.

### STATE OF THE ART

Lissamine green is an organic acidic dye, which is produced synthetically; its staining properties are very similar to those obtained when using rose bengal. However, differently from rose bengal, lissamine green is painless and offers a better colour contrast to vessels and bleeding. Lissamine green selectively stains suffering and dehydrated cells, thus guaranteeing observation of the conjunctival epithelium and highlighting abraded or desquamated epithelial areas.

Lissamine green is typically used, therefore, in vital staining of the external eye surface, not only for the diagnosis of Sjögrens syndrome, which, among many symptoms, also causes ocular dryness (xerophthalmia) characterised by keratoconjunctivitis, but also for detecting ocular dryness due to environmental or age factors or to the use of contact lenses, or for the examination of conjunctival irritations of various origins.

At present, diagnostic tests employing lissamine green are performed by using cardboard strips on one end of which lissamine green solution has been dried. At the time of use, the end of the strip bearing the dye is irrorated with one or two drops of physiological solution, which serves to redissolve the lissamine green. The drop is then let drop into the patient's eye and stains the damaged epithelial areas green.

Said strips are designed to overcome the problem of the reduced water solubility of lissamine green and the consequent high instability of aqueous solutions of lissamine green. The aforesaid strips allow to provide doses of dye in solid form, which are then brought back to a solution form only at the time of use and only in the necessary volume, i.e. one or two drops.

However, these strips show certain drawbacks of a practical nature, as well as of technical nature.

Indeed, since the drop of dye is often released through direct contact between the strip and the eye surface, from the patient's viewpoint, this creates considerable discomfort, in addition to local pain, especially if the surface of the eye is damaged as a result of any of the aforesaid diseases.

Moreover, precisely because lissamine green is not completely water-soluble, the extemporaneous addition of one or two drops thereof to the dry dye does not achieve a constant and measurable concentration of the said dye before use.

The object of the present invention is therefore to offer a practical and effective alternative, which is also well tolerated by patients, to the strips currently in use.

### SUMMARY OF INVENTION

Said object has been achieved by an aqueous solution having a pH of 4.0-6.0 and comprising lissamine green, a buffer, and a stabilizing agent, said stabilizing agent being an ophthalmologically acceptable non-ionic surfactant, as stated in Claim 1.

For the purposes of the present invention, “aqueous solution” is understood as a homogeneous mixture of the various components dissolved in water.

In another aspect, the present invention relates to the use of said aqueous solution for vital staining of the external eye surface.

The characteristics and advantages of the present invention shall become readily apparent from the following detailed description and from the embodiments provided by way of illustrative, non-limiting examples, as the aqueous solution according to the invention has surprisingly shown high stability over time and a notable ease of use.

### DETAILED DESCRIPTION OF THE INVENTION

Therefore, the invention relates to an aqueous ophthalmic solution having a pH of 4.0-6.0 and comprising lissamine green, a buffer, and a stabilizing agent, said stabilizing agent being an ophthalmologically acceptable non-ionic surfactant.

For the purposes of the present invention, the expression “ophthalmologically acceptable non-ionic surfactant” is understood as a non-ionic surfactant suitable for ophthalmologic administration, which is therefore compatible with tissues and fluids present on the eye surface.

Ophthalmologically acceptable non-ionic surfactants are alkyl polyglucosides, octyl glucoside, decyl maltoside, fatty alcohols, cetyl alcohol, oleyl alcohol, cocamide MEA, cocamide DEA, cocamide TEA, C10-C22 alkyl ethoxylates, fatty acid esters of polyols, esters of fatty acid amine oxide, and mixtures thereof. C10-C22 ethoxylated alkyls (e.g., polyoxyl 40 stearate, Myrj 52, Brij 30, Tween 20, Tween 40, Tween 60, Tween 65, Tween 80, polyoxyl 40 hydrogenated castor oil) are preferably chosen from ethoxylates, diethoxylates, triethoxylates, tetraethoxylates, pentaethoxylates or hexaethoxylates of C10-C22 fatty acids, C10-C22 fatty acid amides, C10-C22 fatty amides, C10-C22 alcohols, C10-C22 alkyl phenols, and C10-C22 fatty acid esters of polyols, wherein the hydrogen atom of the terminal hydroxyl group may be, optionally, substituted by a hydrophobic moiety, such as a benzyl, butyl, or methyl group, or mixtures thereof. The fatty acid esters of polyols (e.g., sorbitan monolaurate, sorbitan monooleate) are preferably chosen from C10-C22 fatty acids esters of glycerol, sorbitol, saccharose, polyglucoside, or a mixture thereof. The esters of fatty acid amine oxides are preferably chosen from esters of C10-C22 alkyl dimethyl amine oxides, C10-C22 alkyl diethanolamine oxides, and mixtures thereof.

For the purposes of the present invention, preferred non-ionic surfactants are alkyl polyglucosides, octyl glucoside, C10-C22 fatty acid esters of glycerol, C10-C22 fatty acid esters of sorbitol, C10-C22 fatty acid esters of sucrose, C10-C22 fatty acid esters of polyglucoside, or a mixture thereof.

It has surprisingly been found that the aqueous solution having the characteristics stated above shows a remarkable stability over time, since both the lissamine green titre and the pH remain satisfactory for at least 18 months. Furthermore, an aqueous solution with a predetermined concentration offers the further advantage of being administrable to the eye surface in exactly the same way as eye drops, thereby overcoming the drawbacks observed with the use of cardboard strips.

Indeed, the patient will no longer have the discomfort ascribable to the contact of the end of the cardboard strip with the eye surface, nor will there be any more diagnostic uncertainties ascribable to the variability of the concentration of lissamine green in the drops extemporaneously prepared on the cardboards.

Preferably, said stabilizing agent is in a concentration of up to 2 wt % of the volume of the aqueous solution.

For the purposes of the present invention, “wt % of the volume” or “% w/v” is understood as “grams per 100 ml of aqueous solution”.

More preferably, said stabilizing agent is in a concentration of up to 1 wt % of the volume of the aqueous solution.

In particularly preferred embodiments, the stabilizing agent is in a concentration of 0.001-0.5 wt % of the volume of the aqueous solution.

Preferably, the lissamine green is in a concentration of up to 3 wt % of the volume of the aqueous solution.

More preferably, the lissamine green is in a concentration of 1.0-2.5 wt % of the volume of the aqueous solution.

In particularly preferred embodiments, the lissamine green is in a concentration of 1.3-2.0 wt % of the volume of the aqueous solution.

Preferably, the pH of the aqueous solution is 4.5-5.5.

In particularly preferred embodiments, the pH of the aqueous solution is 4.7-5.2.

In the aqueous solution according to the invention, the buffer serves to maintain the chosen pH.

Preferably, the buffer is in a concentration of up to 5 wt % of the volume of the aqueous solution.

More preferably, the buffer is in a concentration of 1.0-3.0 wt % of the volume of the aqueous solution.

In particularly preferred embodiments, the buffer is in a concentration of 1.5-2.5 wt % of the volume of the aqueous solution.

Preferably, said buffer is citric acid-sodium citrate, acetic acid-sodium acetate, boric acid-sodium borate, citric acid-disodium hydrogen phosphate (also known as ‘McIlvaine buffer’), citric acid-monopotassium phosphate-boric acid-diethyl barbituric acid, TRIS-borate, or a combination thereof.

In particularly preferred embodiments, the buffer is citric acid-sodium citrate.

In particularly preferred embodiments, the lissamine green is the only active ingredient for the vital staining of the external eye surface which is present in the aqueous solution.

In further particularly preferred embodiments of the aqueous solution according to the invention, water it is the only solvent present therein.

In still further particularly preferred embodiments, the aqueous solution according to the invention does not comprise preservatives or others dyes in addition to lissamine green.

Preferably, the lissamine green is in a higher concentration than the stabilizing agent.

In preferred embodiments, the concentrations of lissamine green and stabilizing agent are in a ratio of 200:1 to 2:1, more preferably 50:1 to 5:1, and even more preferably 30:1 to 10:1.

In further embodiments, the aqueous solution according to the invention is in the form of a unit dose.

Preferably, said unit dose comprises 0.1-2.0 ml of aqueous solution as disclosed above.

In particularly preferred embodiments, said unit dose comprises 0.2-1.0 ml of aqueous solution as disclosed above.

It should be understood that the preferred aspects specified for the individual components of the aqueous solution, should likewise be considered as preferred in the unit dose embodiments stated above.

The aqueous solutions which are preferred, and likewise the unit doses, are those having a pH of 4.5-5.5 and comprising:
up to 2% w/v of stabilizing agent,
up to 3% w/v of lissamine green,
up to 5% w/v of buffer, and
water.

The aqueous solutions which are more preferred, and likewise the unit doses, are those having a pH of 4.5-5.5 and comprising:
up to 1% w/v of stabilizing agent,
1.0-2.5% w/v of lissamine green,
1.0-3.0% w/v of buffer, and
water.

The aqueous solutions which are even more preferred, and likewise the unit doses, are those having a pH of 4.5-5.5 and comprising:
0.001-0.5% w/v of stabilizing agent,
1.3-2.0% w/v of lissamine green,
1.5-2.5% w/v of buffer, and
water.

The aqueous solution according to the invention, also in the form of a unit dose, may further comprise ophthalmologically acceptable excipients. The term “ophthalmologically acceptable excipient” should be understood as a compound or mixture which is suitable for use in a formulation for administration to the external eye surface. For example, an excipient of this type should generally not cause an adverse response in a patient, nor should it significantly inhibit the action of the lissamine green on the eye surface.

Suitable excipients are antioxidants, gelling agents, sequestrants, binders, lubricants, thickening agent, tonicity regulators, filmogenic substances, and mixtures thereof.

Thus, suitable antioxidants are sodium metabisulphite, sodium thiosulphate, ascorbic acid, sodium ascorbate, glucose, cysteine, and mixtures thereof.

Suitable sequestrants are EDTA and monosodium, disodium, and potassium salts thereof, diethylene triamine pentamethylene phosphonic acid, hexamethylene diamine tetramethylene phosphonic acid, ethylene diamine tetramethylene phosphonic acid, amino trimethylene phosphonates and mixtures thereof.

Preferably, the sequestrants are EDTA, monosodium, disodium, and potassium salts thereof, and mixtures thereof.

Suitable thickening agents are TS polysaccharide, PVP, carboxyvinyl polymers, xanthan gum, sodium hyaluronate, hydroxyethyl cellulose (HEC), hydroxypropyl methylcellulose (HPMC), polyethylene glycol, mucopolysaccharides, cellulose and the esters thereof, natural gums and the esters thereof, pectins, polyacrylates, and mixtures thereof.

Preferably, the thickening agents are TS polysaccharide, mucopolysaccharides, and mixtures thereof.

Tonicity regulators are inorganic salts, such as sodium chloride, potassium chloride, magnesium chloride, and calcium chloride, or polyols or sugars, such as glycerol, propylene glycol, erythritol, mannitol, sorbitol, and trehalose, or amino acids, such as carnitine and betaine.

In preferred embodiments, the aqueous solution according to the invention has a pH of 4.8-5.1 and comprises:
0.01 to 0.3% w/v of alkyl polyglucosides,
1.4-1.8% w/v of lissamine green,
1.5-2.5% w/v of citric acid-sodium citrate buffer, and
water.

In more preferred embodiments, the aqueous solution according to the invention has a pH of 4.8-5.1 and comprises:
0.01 to 0.3% w/v of alkyl polyglucosides,
1.4-1.8% w/v of lissamine green,
1.5-2.5% w/v of citric acid-sodium citrate buffer,
0.01-0.5% w/v of sodium metabisulphite,
0.01-0.2% w/v of EDTA,
0.01-0.3% w/v of TS polysaccharide, and
water.

In some embodiments, the aqueous solution according to the invention has a pH of 4.0-6.0 and consists essentially of lissamine green, a buffer, water, and a stabilizing agent, said stabilizing agent being an ophthalmologically acceptable non-ionic surfactant. The expression “consisting essentially of” means that the lissamine is the only active ingredient for the vital staining of the external eye surface which is present in the aqueous solution according to the invention, while further components or excipients do not interfere with the action of the lissamine green and are water-miscible and soluble.

In other embodiments, the aqueous solution according to the invention has a pH of 4.0-6.0 and consists of lissamine green, a buffer, water, and a stabilizing agent, said stabilizing agent being an ophthalmologically acceptable non-ionic surfactant, and ophthalmologically acceptable excipients.

The aqueous solution according to the present invention may be prepared by using known methods. Indeed, for administration to the external eye surface, the components may, for example, be mixed as they are or with one or more excipients, added one after the other, under stirring.

Typically, the aqueous solutions of the present invention are sterilised prior to use according to known methods, e.g. by a gamma ray treatment.

In another aspect, the present invention relates to the use of the aqueous solution disclosed above for vital staining of the external eye surface. As already mentioned, indeed, the aqueous solution according to the invention has surprisingly overcome the drawbacks associated with the use of cardboard strips bearing dried lissamine green.

Therefore, the aqueous solution according to the invention is therefore usable in the ophthalmic field as it is well tolerated by the eye and therefore finds advantageous application in the diagnosis of corneal and conjunctival anomalies.

Preferably, the aqueous solution according to the invention is administered to the external eye surface in an amount of 0.005-0.050 ml, preferably 0.010-0.015 ml. It should be understood that all the possible combinations of the preferred aspects of the components of the aqueous solution, the unit doses, the preparation, and the uses of said aqueous solution are described herein and therefore are also preferred.

It should also be understood that all aspects identified as favourable and advantageous for the aqueous solution and the components thereof, should be deemed equally preferable and advantageous also for the preparation and the uses of said aqueous solution.

Below are working examples of the present invention provided for illustrative purposes.

### EXAMPLES

### Example 1

The following aqueous solution with a pH of 4.9 was prepared:

| | % w/v |
|---|---|
| Lissamine green | 1.65% |
| Tribasic sodium citrate | 1.36% |
| Citric acid | 0.50% |
| TRITON ™ CG-50 (*) | 0.10% |
| Distilled water | balance to 100% |

(*) alkyl-polyglucoside non-ionic surfactant (Dow Chemical Company)

### Example 2

Two aqueous solutions were prepared for comparison:

| | A | B |
|---|---|---|
| | % w/v | % w/v |
| Lissamine green | 1.50% | 1.50% |
| Tribasic sodium citrate | 0.50% | 0.50% |
| Citric acid | 0.03% | 0.03% |
| Distilled water | balance to 100% | balance to 100% |
| pH | 5.5 | 6.5 |

### Example 3

The following aqueous solution with a pH of 4.9 was prepared:

| | % w/v |
|---|---|
| Lissamine green | 1.65% |
| Tribasic sodium citrate | 1.36% |
| Citric acid | 0.50% |
| Sodium metabisulphite | 0.20% |
| EDTA | 0.05% |
| TRITON ™ CG-50 | 0.10% |
| TS polysaccharide | 0.10% |
| Distilled water | balance to 100% |

### Example 4

The following aqueous solution with a pH of 5.0 was prepared:

| | % w/v |
|---|---|
| Lissamine green | 1.50% |
| Tribasic sodium citrate | 0.50% |
| Citric acid | 0.04% |
| Sodium chloride | 0.40% |
| Sodium metabisulphite | 0.20% |
| EDTA | 0.05% |
| TRITON ™ CG-50 | 0.10% |
| TS polysaccharide | 0.1% |
| Distilled water | balance to 100% |

### Example 5

The following aqueous solution with apH of 5.0 was prepared:

| | % w/v |
|---|---|
| Lissamine green | 1.5% |
| Tribasic sodium citrate | 0.5% |
| Citric acid | 0.04% |
| Sodium chloride | 0.3% |
| Sodium metabisulphite | 0.2% |
| EDTA | 0.05% |
| Triton | 0.1% |
| Trehalose | 1.0% |
| PVP | 3.0% |
| Distilled water | balance to 100% |

### Example 6

Test of Stability Over Time

The aqueous solutions A and B used for comparison in Example 2 were tested with the same composition but different pHs.

Samples of both solutions were subjected to two separate stability studies:
the first set of samples of solutions A and B was stored at 25° C. (RH 60%),
the second set of samples of solutions A and B was stored at 40° C. (RH 74%).

After six months in the conditions stated above, all the samples showed a severe decline in the green lissamine titre, accompanied by a progressive acidification and the solutions, which were originally an intense blue colour, tended towards green.

Samples of the aqueous solution in Example 1 were subjected to a stability study for a total of nine months at 25° C. (RH 60%).

After the first two months, during which time a decrease of approximately 10-12% was observed in the lissamine green titre, the said titre then remained nearly stable until the end of the observation period.

The pH also showed a similar trend, with a decrease of approximately 6% in absolute value over the first two months, followed by a stable level until the end of the observation period.

1. An aqueous solution having pH of 4.0-6.0 and comprising lissamine green, a buffer and a stabilizing agent, said stabilizing agent being a non-ionic surfactant ophthalmologically acceptable.

2. The aqueous solution of claim 1, wherein said stabilizing agent is alkyl polyglucoside, octyl glucoside, decyl maltoside, fatty alcohol, cetyl alcohol, oleyl alcohol, cocamide MEA, cocamide DEA, cocamide TEA, ethoxylate, ester of a fatty acid of polyol or oxide of amines, or a mixture thereof.

3. The aqueous solution of claim 2, wherein said stabilizing agent is alkyl polyglucoside, octyl glucoside, ester of C10-C22 fatty acid of glycerol, sorbitol, sucrose or polyglucoside, or a mixture thereof.

4. The aqueous solution of claim 1, wherein said stabilizing agent is in a concentration up to 2% by weight on volume of the aqueous solution.

5. The aqueous solution of claim 1, wherein lissamine green is in a concentration up to 3% by weight on volume of the aqueous solution.

6. The aqueous solution of claim 1, wherein said buffer is citric acid-sodium citrate, acetic acid-sodium acetate, sodium borate-boric acid, citric acid-disodium hydrogen phosphate, citric acid-monopotassium phosphate-boric acid-diethylbarbituric acid, TRIS-borate, or a combination thereof.

7. The aqueous solution of claim 1, wherein lissamine green is the only active ingredient for the vital staining of the external eye surface which is present in the aqueous solution.

8. The aqueous solution of claim 1, wherein the concentrations of lissamine green and stabilizing agent are in a ratio of 200:1 to 2:1.

9. The aqueous solution of claim 1, having a pH 4.8-5.1 and comprising:
0.01 to 0.3% w/v of alkyl polyglucosides,
1.4-1.8% w/v of lissamine green,
1.5-2.5% w/v of citric acid-sodium citrate buffer, and
water.

10. (canceled)

11. The aqueous solution of claim 1, wherein water it is the only solvent present in the aqueous solution.

12. The aqueous solution of claim 1, wherein the pH of the aqueous solution is 4.7-5.2.

13. The aqueous solution of claim 1, wherein the aqueous solution does not comprise preservatives or other dyes in addition to lissamine green.

14. The aqueous solution of claim 1, wherein the aqueous solution is in the form of a unit dose comprising 0.1-2.0 ml of the aqueous solution.

15. The aqueous solution of claim 1, having a pH of 4.5-5.5 and comprising:
up to 2% w/v of stabilizing agent,
up to 3% w/v of lissamine green,
up to 5% w/v of buffer, and
water.

16. The aqueous solution of claim 15, having a pH of 4.5-5.5 and comprising:
up to 1% w/v of stabilizing agent,
1.0-2.5% w/v of lissamine green,
1.0-3.0% w/v of buffer, and
water.

17. The aqueous solution of claim 16, having a pH of 4.5-5.5 and comprising:
0.001-0.5% w/v of stabilizing agent,
1.3-2.0% w/v of lissamine green,
1.5-2.5% w/v of buffer, and
water.

18. The aqueous solution of claim 17, having a pH of 4.8-5.1 and comprising:
0.01 to 0.3% w/v of alkyl polyglucosides,
1.4-1.8% w/v of lissamine green,
1.5-2.5% w/v of citric acid-sodium citrate buffer,
0.01-0.5% w/v of sodium metabisulphite,
0.01-0.2% w/v of EDTA,
0.01-0.3% w/v of TS polysaccharide, and
water.

19. The aqueous solution of claim 4, wherein said stabilizing agent is in a concentration up to 1% by weight on volume of the aqueous solution.

20. The aqueous solution of claim 5, wherein lissamine green is in a concentration of 1.0-2.5% by weight on the volume of the solution aqueous.
